(19) United States
(12) Patent Application Publication
(10) Pub. No.: US 2022/0178894 A1
(43) Pub. Date: Jun. 9, 2022

(54) FORMALDEHYDE CONCENTRATION MEASUREMENT METHOD AND APPARATUS, AND AIR PURIFIER

(71) Applicant: GREE ELECTRIC APPLIANCES, INC. OF ZHUHAI, Qianshan, Zhuhai, Guangdong (CN)

(72) Inventors: Zhengwei KANG, Zhuhai (CN); Linhai NI, Zhuhai (CN); Bin QIN, Zhuhai (CN); Weibang SU, Zhuhai (CN)

(21) Appl. No.: 17/601,901

(22) PCT Filed: Dec. 18, 2019
(86) PCT No.: PCT/CN2019/126167

§ 371 Date: Oct. 6, 2021

(30) Foreign Application Priority Data
Jun. 21, 2019 (CN) 201910544470.7

(51) Int. Cl. G01N 33/00

(52) U.S. Cl. CPC G01N 33/0006; G01N 33/006; G01N 33/0047

(57) ABSTRACT

Formaldehyde concentration measurement method and apparatus, and an air purifier. The formaldehyde concentration measurement method includes: determining an initial formaldehyde concentration value measured by a formaldehyde sensor; acquiring temperature information and/or humidity information of an environment surrounding the formaldehyde sensor; determining a formaldehyde concentration compensation value of the formaldehyde sensor according to the temperature information and/or the humidity information; determining a target formaldehyde concentration value of the formaldehyde sensor according to the formaldehyde concentration compensation value and the initial formaldehyde concentration value.

### CROSS-REFERENCE TO RELATED APPLICATION

The present disclosure claims priority to Chinese Patent Application No. 201910544470.7, entitled “FORMALDEHYDE CONCENTRATION MEASUREMENT METHOD AND APPARATUS, AND AIR PURIFIER”, filed on Jun. 21, 2019, the contents of which are herein incorporated by reference in its entirety.

### FIELD

The present disclosure relates to the technical field of measurement equipment, and in particular to a formaldehyde concentration measurement method and apparatus, and an air purifier.

### BACKGROUND

Air quality measurement equipment is usually equipped with an air quality sensor which is used to measure air quality in the surrounding environment. For example, a formaldehyde sensor is used to measure the content of formaldehyde gas in the air to obtain a formaldehyde concentration value. With the operation of the formaldehyde sensor, due to the changes of factors such as temperature and humidity in the surrounding environment, the formaldehyde concentration values measured by the formaldehyde sensor in the environments with different temperatures and humidity levels are different, resulting in errors of the formaldehyde concentration value measured in different environments.

### SUMMARY

In view of the above, the present disclosure provides a formaldehyde concentration measurement method and apparatus, and an air purifier, which are capable of reducing errors of formaldehyde concentration value measured by a formaldehyde sensor in different environments, thereby improving the measurement accuracy of the formaldehyde sensor.

In view of this, in a first aspect, the present disclosure provides a formaldehyde concentration measurement method, including steps of:
determining an initial formaldehyde concentration value measured by a formaldehyde sensor;
acquiring temperature information and/or humidity information of an environment surrounding the formaldehyde sensor;
determining a formaldehyde concentration compensation value of the formaldehyde sensor according to the temperature information and/or the humidity information; and
determining a target formaldehyde concentration value of the formaldehyde sensor according to the formaldehyde concentration compensation value and the initial formaldehyde concentration value.

With reference to the first aspect, in some embodiments, the temperature information is a temperature value, the humidity information is a humidity value, and the step of determining the formaldehyde concentration compensation value of the formaldehyde sensor according to the temperature information and/or the humidity information includes steps of:

calculating a first difference between the temperature value and a set temperature threshold, and/or calculating a second difference between the humidity value and a set humidity threshold; and
determining the formaldehyde concentration compensation value of the formaldehyde sensor according to the first difference and/or the second difference.

With reference to the first aspect, in some embodiments, the step of determining the formaldehyde concentration compensation value of the formaldehyde sensor according to the first difference and/or the second difference includes steps of:
determining a first formaldehyde concentration compensation value of the formaldehyde sensor corresponding to the first difference between the temperature value and the set temperature threshold, according to a preset corresponding relationship between the difference, between the temperature value and the set temperature threshold, and the formaldehyde concentration compensation value of the formaldehyde sensor;
and/or
determining a second formaldehyde concentration compensation value of the formaldehyde sensor corresponding to the second difference between the humidity value and the set humidity threshold, according to a preset corresponding relationship between the difference, between the humidity value and the set humidity threshold, and the formaldehyde concentration compensation value of the formaldehyde sensor; and
setting the first formaldehyde concentration compensation value, or the second formaldehyde concentration compensation value, or a sum of the first formaldehyde concentration compensation value and the second formaldehyde concentration compensation value, as the formaldehyde concentration compensation value of the formaldehyde sensor.

With reference to the first aspect, in some embodiments, the method further includes steps of:
acquiring operating parameter information of the formaldehyde sensor in a preset time period, wherein the operating parameter information includes: total operating time period of the formaldehyde sensor in the preset time period, and/or total number of operations of the formaldehyde sensor in the preset time period; and
sending a corresponding command signal to the formaldehyde sensor in order to instruct the formaldehyde sensor to perform zero point calibration when the operating parameter information satisfies a preset condition.

With reference to the first aspect, in some embodiments, the step of sending the corresponding command signal to the formaldehyde sensor in order to instruct the formaldehyde sensor to perform the zero point calibration, when the operating parameter information satisfies the preset condition, includes steps of:
comparing the total operating time period of the formaldehyde sensor in the preset time period with a set total operating time period threshold to obtain a first comparison result; and/or
comparing the total number of operations of the formaldehyde sensor in the preset time period with a set total number threshold of operations to obtain a second comparison result;
determining that the operating parameter information satisfies the preset condition when the first comparison result is: the total operating time period of the formaldehyde sensor in the preset time period is greater than the set total operating time period threshold, and/or the second comparison result is: the total number of operations of the formaldehyde sensor in the preset time period is greater than the set total number threshold of operations.

In a second aspect, the present disclosure provides a formaldehyde concentration measurement apparatus, including a processor, a communication interface, a formaldehyde sensor, and a temperature sensor and/or a humidity sensor; the formaldehyde sensor and the temperature sensor and/or the humidity sensor are electrically connected to the processor via the communication interface.

The formaldehyde sensor is configured to measure a concentration of formaldehyde to obtain an initial formaldehyde concentration value, and send the initial formaldehyde concentration value to the processor.

The temperature sensor is configured to collect temperature information of an environment surrounding the formaldehyde sensor, and send the temperature information to the processor.

The temperature sensor is configured to collect humidity information of the environment surrounding the formaldehyde sensor, and send the humidity information to the processor.

The processor is configured to determine a formaldehyde concentration compensation value of the formaldehyde sensor according to the received temperature information and/or humidity information, and to determine a target formaldehyde concentration value of the formaldehyde sensor according to the formaldehyde concentration compensation value and the received initial formaldehyde concentration value.

With reference to the second aspect, in some embodiments, the temperature information is a temperature value, the humidity information is a humidity value, and the processor includes:
a difference calculation module configured to calculate a first difference between the temperature value and a set temperature threshold, and/or calculating a second difference between the humidity value and a set humidity threshold; and
a compensation value determination module configured to determine the formaldehyde concentration compensation value of the formaldehyde sensor according to the first difference and/or the second difference calculated by the difference calculation module.

With reference to the second aspect, in some embodiments, the compensation value determination module includes:
a first compensation value determination unit configured to determine a first formaldehyde concentration compensation value of the formaldehyde sensor corresponding to the first difference between the temperature value and the set temperature threshold, according to a preset corresponding relationship between the difference, between the temperature value and the set temperature threshold, and the formaldehyde concentration compensation value of the formaldehyde sensor; and/or
a second compensation value determination unit configured to determine a second formaldehyde concentration compensation value of the formaldehyde sensor corresponding to the second difference between the humidity value and the set humidity threshold, according to a preset corresponding relationship between the difference, between the humidity value and the set humidity threshold, and the formaldehyde concentration compensation value of the formaldehyde sensor; and
a formaldehyde concentration compensation value determination unit configured to set the first formaldehyde concentration compensation value determined by the first compensation value determination unit, or the second formaldehyde concentration compensation value determined by the second compensation value determination unit, or a sum of the first formaldehyde concentration compensation value determined by the first compensation value determination unit and the second formaldehyde concentration compensation value determined by the second compensation value determination unit, as the formaldehyde concentration compensation value of the formaldehyde sensor.

With reference to the second aspect, in some embodiments, the processor further includes:
a parameter information acquisition module configured to acquire operating parameter information of the formaldehyde sensor in a preset time period. The operating parameter information includes: total operating time period of the formaldehyde sensor in the preset time period, and/or total number of operations of the formaldehyde sensor in the preset time period.

The processor sends a corresponding command signal to the formaldehyde sensor in order to instruct the formaldehyde sensor to perform zero point calibration when the operating parameter information acquired by the parameter information acquisition module satisfies a preset condition.

With reference to the second aspect, in some embodiments, the processor further includes:

a first comparison module configured to compare the total operating time period of the formaldehyde sensor in the preset time period with a set total operating time period threshold to obtain a first comparison result; and/or
a second comparison module configured to compare the total number of operations of the formaldehyde sensor in the preset time period with a set total number threshold of operations to obtain a second comparison result.

The processor determines that the operating parameter information acquired by the parameter information acquisition module satisfies the preset condition, on a condition that the first comparison result obtained by the first comparison module is: the total operating time period of the formaldehyde sensor in the preset time period is greater than the set total operating time period threshold, and/or on a condition that the second comparison result obtained by the second comparison module is: the total number of operations of the formaldehyde sensor in the preset time period is greater than the set total number threshold of operations.

In a third aspect, the present disclosure provides an air purifier, including the formaldehyde concentration measurement apparatus as described in the second aspect.

The embodiments of the present disclosure provide a formaldehyde concentration measurement method and apparatus, and an air purifier. The formaldehyde concentration measurement method includes steps of: determining an initial formaldehyde concentration value measured by a formaldehyde sensor; acquiring temperature information and/or humidity information of an environment surrounding the formaldehyde sensor; determining a formaldehyde concentration compensation value of the formaldehyde sensor according to the temperature information and/or the humidity information; and determining a target formaldehyde concentration value of the formaldehyde sensor according to the formaldehyde concentration compensation value and the initial formaldehyde concentration value.

According to the formaldehyde concentration measurement method, errors of the formaldehyde concentration value measured by the formaldehyde sensor in different environments can be reduced, thereby improving the measurement accuracy of the formaldehyde sensor. For example, the formaldehyde concentration compensation value of the formaldehyde sensor is calculated according to the temperature information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different temperature environments. In another example, the formaldehyde concentration compensation value of the formaldehyde sensor is calculated according to the humidity information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different humidity environments. In yet another example, the formaldehyde concentration compensation value of the formaldehyde sensor is calculated according to the temperature and humidity information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different temperature and humidity environments.

### BRIEF DESCRIPTION OF THE DRAWINGS

In order to more clearly illustrate the technical solutions in the embodiments of the present disclosure or in the prior art, the drawings that are to be referred in the description of the embodiments or the prior art are briefly described below. Obviously, the drawings in the following description are only show embodiments of the present disclosure. For those of ordinary skill in the art, other drawings can be obtained according to the disclosed drawings without any creative efforts.

FIG. 1 is a schematic flow chart of a formaldehyde concentration measurement method provided in some embodiments of the present disclosure.

FIG. 2 is a schematic flow chart of another formaldehyde concentration measurement method provided in some embodiments of the present disclosure.

FIG. 3 is a schematic flow chart of yet another formaldehyde concentration measurement method provided in some embodiments of the present disclosure.

FIG. 4 is a schematic structural view of a formaldehyde concentration measurement apparatus provided in some embodiments of the present disclosure.

### DETAILED DESCRIPTION

In order to make the objective, the technical solutions, and the merits of the embodiments of the present disclosure clear, the technical solutions in the embodiments of the present disclosure are clearly and completely described below in conjunction with the drawings in the embodiments of the present disclosure. Obviously, the embodiments described are only some, rather than all, of the embodiments of the present disclosure. All other embodiments obtained by a person of ordinary skill in the art based on the embodiments in the present disclosure without creative efforts are within the scope of the present disclosure.

A server implementing various embodiments of the present disclosure will now be described with reference to the accompanying drawings. In the following description, the use of end words such as “module”, “component” or “unit” for indicating elements is only for facilitating the description of the present disclosure, and has no specific meaning in itself. Therefore, “module” and “component” may be used interchangeably.

As shown in FIG. 1, some embodiments of the present disclosure provide a formaldehyde concentration measurement method, including the following steps.

At step S101, an initial formaldehyde concentration value measured by a formaldehyde sensor is determined.

The embodiments of the present disclosure are applied to the technical field of measurement equipment. Specifically, when the formaldehyde sensor in the air purifier is used to measure the formaldehyde content in the air, the formaldehyde concentration value measured in environments with different temperatures and humidity levels can be corrected according to the temperature information and the humidity information of the environment surrounding the formaldehyde sensor, so as to reduce measurement errors of the formaldehyde sensor in different environments. The embodiments of the present disclosure can also be applied to the field that air quality is measured by using other air quality sensors in the air quality detection equipment, which is not limited herein.

The initial formaldehyde concentration value in some embodiments of the present disclosure is an initial measurement value of the formaldehyde concentration obtained by measuring the formaldehyde content in the air by the formaldehyde sensor in the current temperature and humidity environment, i.e., an initial measurement value that has not been corrected according to the temperature and humidity information of the surrounding environment.

At step S102, temperature information and/or humidity information of the environment surrounding the formaldehyde sensor is acquired.

In some embodiments of the present disclosure, the temperature information is a temperature value, and the humidity information is a humidity value. Both the temperature value and the humidity value are environmental factors that affect the initial formaldehyde concentration value measured by the formaldehyde sensor. In an embodiment, only one environmental factor, e.g., the temperature value, may be considered, and the formaldehyde concentration compensation value of the formaldehyde sensor is calculated according to the temperature value. In another embodiment, only another environmental factor, e.g., the humidity value, may be considered, and the formaldehyde concentration compensation value of the formaldehyde sensor is calculated according to the humidity value. In yet another embodiment, the two environmental factors, i.e., the temperature value and the humidity value, may be considered simultaneously, and the formaldehyde concentration compensation value of the formaldehyde sensor is calculated according to the temperature value and the humidity value.

In some embodiments of the present disclosure, the temperature value of the environment surrounding the formaldehyde sensor is measured by a temperature sensor, and the humidity value of the environment surrounding the formaldehyde sensor is measured by a humidity sensor.

At step S103, a formaldehyde concentration compensation value of the formaldehyde sensor is determined according to the temperature information and/or the humidity information.

The formaldehyde concentration compensation value calculated in some embodiments of the present disclosure is used to correct the initial formaldehyde concentration value measured by the formaldehyde sensor. The formaldehyde concentration compensation value may be a positive value or a negative value. The specific calculation of the formaldehyde concentration compensation value is illustrated in the following steps.

At step S104, a target formaldehyde concentration value of the formaldehyde sensor is determined according to the formaldehyde concentration compensation value and the initial formaldehyde concentration value.

In some embodiments of the present disclosure, corresponding formaldehyde concentration compensation values are calculated according to the temperature values of the surrounding environment in different temperatures and the humidity values of the surrounding environment in different humidity levels, and the initial formaldehyde concentration value with large error and low accuracy is corrected by using the formaldehyde concentration compensation value, so as to obtain the target formaldehyde concentration value with smaller error and higher accuracy.

According to the formaldehyde concentration measurement method provided in some embodiments of the present disclosure, errors of the formaldehyde concentration value measured by the formaldehyde sensor in different environments can be reduced, thereby improving the measurement accuracy of the formaldehyde sensor. For example, the formaldehyde concentration compensation value of the formaldehyde sensor is calculated according to the temperature information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different temperature environments. In another example, the formaldehyde concentration compensation value of the formaldehyde sensor is calculated according to the humidity information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different humidity environments. In yet another example, the formaldehyde concentration compensation value of the formaldehyde sensor is calculated according to the temperature and humidity information of the surrounding environment, and the initial formaldehyde concentration value measured by the formaldehyde sensor is corrected by using the formaldehyde concentration compensation value, so as to reduce the errors of the formaldehyde concentration value measured by the formaldehyde sensor in different temperature and humidity environments.

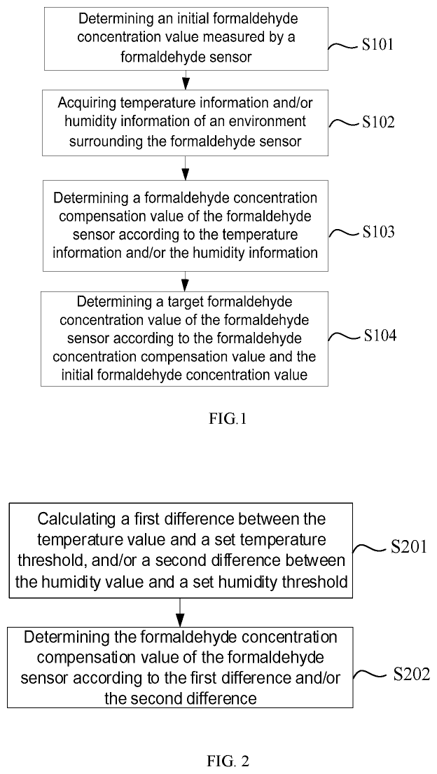

Referring to FIG. 2, some embodiments of the present disclosure also provide a formaldehyde concentration measurement method. Based on the embodiment shown in FIG. 1, the step S103, determining the formaldehyde concentration compensation value of the formaldehyde sensor according to the temperature information and/or the humidity information, further includes the following steps.

At step S201, a first difference between the temperature value and a set temperature threshold is calculated, and/or a second difference between the humidity value and a set humidity threshold is calculated.

In some embodiments of the present disclosure, the set temperature threshold includes but is not limited to 25 degrees, and the set humidity threshold includes but is not limited to 50%. For example, in an environment where the temperature value a is 25 degrees, the formaldehyde concentration in the air measured by the formaldehyde sensor is 1.2 mg/m³, i.e., the initial formaldehyde concentration value A measured in the environment where the temperature value a is 25 degrees is 1.2 mg/m³. The temperature threshold is subtracted from the temperature value a (25 degrees−25 degrees), and the difference between the temperature value a and the temperature threshold is 0, then the formaldehyde concentration compensation value corresponding to the difference is also 0. Thus, there is no need to correct the initial formaldehyde concentration value A, and the initial formaldehyde concentration value A is the target formaldehyde concentration value of the formaldehyde sensor.

In another example, in an environment where the temperature value b is 6 degrees, the formaldehyde concentration in the air measured by the formaldehyde sensor is 1 mg/m³, i.e., the initial formaldehyde concentration value B measured in the environment where the temperature value b is 6 degrees is 1 mg/m³. The temperature threshold is subtracted from the temperature value b (6 degrees−25 degrees), and the difference between the temperature value b and the temperature threshold is obtained as −19 degrees, then the formaldehyde concentration compensation value corresponding to the difference (−19 degrees) is 0.2 mg/m³. The formaldehyde concentration compensation value (0.2 mg/m³) and the initial formaldehyde concentration value B (1 mg/m³) are added together to obtain the target formaldehyde concentration value (1.2 mg/m³).

At step S202, the formaldehyde concentration compensation value of the formaldehyde sensor is determined according to the first difference and/or the second difference.

In some embodiments of the present disclosure, the step S202 of determining the formaldehyde concentration compensation value of the formaldehyde sensor according to the first difference and/or the second difference includes steps of:
determining a first formaldehyde concentration compensation value of the formaldehyde sensor corresponding to the first difference between the temperature value and the set temperature threshold, according to a preset corresponding relationship between the difference, between the temperature value and the set temperature threshold, and the formaldehyde concentration compensation value of the formaldehyde sensor; and/or
determining a second formaldehyde concentration compensation value of the formaldehyde sensor corresponding to the second difference between the humidity value and the set humidity threshold, according to a preset corresponding relationship between the difference, between the humidity value and the set humidity threshold, and the formaldehyde concentration compensation value of the formaldehyde sensor; and
setting the first formaldehyde concentration compensation value, or the second formaldehyde concentration compensation value, or a sum of the first formaldehyde concentration compensation value and the second formaldehyde concentration compensation value, as the formaldehyde concentration compensation value of the formaldehyde sensor.

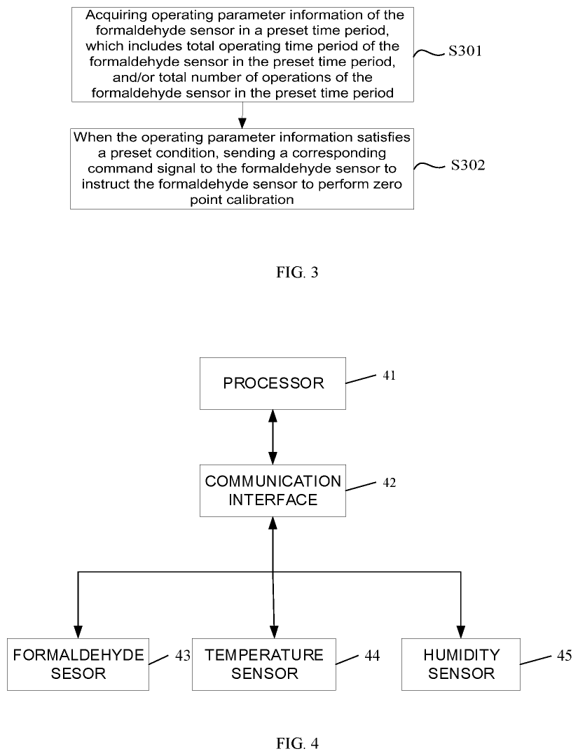

There is a problem that the accuracy of the measured formaldehyde concentration becomes relatively low when the formaldehyde sensor has been operated for a long time. Referring to FIG. 3, some embodiments of the present disclosure also provide a formaldehyde concentration measurement method. Based on the embodiment shown in FIG. 1, the method further includes the following steps.

At step S301, operating parameter information of the formaldehyde sensor in a preset time period is acquired, wherein the operating parameter information includes: total operating time period of the formaldehyde sensor in the preset time period, and/or total number of operations of the formaldehyde sensor in the preset time period.

In some embodiments of the present disclosure, the operating parameter information is the information used to describe the operating conditions of the formaldehyde sensor. The operating parameter information can be the total operating time period of the formaldehyde sensor in a preset time period, or the operating parameter information can be the total number of operations of the formaldehyde sensor in a preset time period, or the operating parameter information can include the total operating time period and the total number of operations of the formaldehyde sensor in a preset time period.

Under different formaldehyde concentrations, the total operating time periods of the formaldehyde sensor in the preset time period are different, and the total numbers of operations of the formaldehyde sensor in the preset time period are also different. The higher the formaldehyde concentration, the longer the total operating time period of the formaldehyde sensor in the preset time period, similarly, the larger the total number of operations of the formaldehyde sensor in the preset time period.

For example, in the case where the preset time period is set to 1 hour, when the formaldehyde concentration>a high concentration value N1, the total operating time period of the formaldehyde sensor within 1 hour is recorded as T1 hours;

When medium concentration N2<the formaldehyde concentration<high concentration N1, the total operating time period of the formaldehyde sensor within 1 hour is recorded as T2 hours;

When low concentration N3<the formaldehyde concentration<medium concentration N2, the total operating time period of the formaldehyde sensor within 1 hour is recorded as T3 hours;

When 0<the formaldehyde concentration<low concentration N3, the total operating time period of the formaldehyde sensor within 1 hour is recorded as T4 hours;

Then the relationship among the total operating time periods is: T4<T3<T2<T1.

For example, in the case where the preset time period is set to 1 hour, when the formaldehyde concentration>high concentration value N1, the total number of operations of the formaldehyde sensor within 1 hour is recorded as a1 times;

When medium concentration N2<the formaldehyde concentration<high concentration N1, the total number of operations of the formaldehyde sensor within 1 hour is recorded as a2 times;

When low concentration N3<the formaldehyde concentration<medium concentration N2, the total number of operations of the formaldehyde sensor within 1 hour is recorded as a3 times;

When 0<the formaldehyde concentration<low concentration N3, the total number of operations of the formaldehyde sensor within 1 hour is recorded as a4 times;

Then the relationship among total operating time is: a4<a3<a2<a1.

At step S302, when the operating parameter information satisfies a preset condition, a corresponding command signal is sent to the formaldehyde sensor in order to instruct the formaldehyde sensor to perform zero point calibration.

In other embodiments of the present disclosure, the step S302, sending the corresponding command signal to the formaldehyde sensor in order to instruct the formaldehyde sensor to perform the zero point calibration, when the operating parameter information satisfies the preset condition, includes steps of:
comparing the total operating time period of the formaldehyde sensor in a preset time period with a set total operating time period threshold to obtain a first comparison result;
and/or
comparing the total number of operations of the formaldehyde sensor in a preset time period with a set total number threshold of operations to obtain a second comparison result; and
determining that the operating parameter information satisfies the preset condition when the first comparison result is: the total operating time period of the formaldehyde sensor in the preset time period is greater than the set total operating time period threshold, and/or the second comparison result is: the total number of operations of the formaldehyde sensor in the preset time period is greater than the set total number threshold of operations.

In some embodiments of the present disclosure, the total operating time period threshold can be set to 30 minutes, and the total number threshold of operations can be set to 15 times, which are not limited in the embodiments of the present disclosure.

In some embodiments of the present disclosure, by counting the total operating time period and/or the total number of operations of the formaldehyde sensor in a preset time period, and sending command signals to the formaldehyde sensor periodically by a processor to allow the formaldehyde sensor to re-calibrate the zero point, the self-calibration of the formaldehyde sensor can be realized, the problem of zero point drift caused by the long-term operation of the formaldehyde sensor is solved, and the accuracy of the formaldehyde concentration measured by the formaldehyde sensor is improved.

Referring to FIG. 4, some embodiments of the present disclosure provide a formaldehyde concentration measurement apparatus including a processor 41, a communication interface 42, a formaldehyde sensor 43, and a temperature sensor 44 and/or a humidity sensor 45. The formaldehyde sensor 43 and the temperature sensor 44 and/or the humidity sensor 45 are electrically connected to the processor 41 via the communication interface 42.

The formaldehyde sensor 43 is configured to measure the formaldehyde concentration to obtain an initial formaldehyde concentration value, and send the initial formaldehyde concentration value to the processor 41.

The temperature sensor 44 is configured to collect temperature information of the environment surrounding the formaldehyde sensor 43, and send the temperature information to the processor 41.

The temperature sensor 45 is configured to collect humidity information of the environment surrounding the formaldehyde sensor 43, and send the humidity information to the processor 41.

The processor 41 is configured to determine a formaldehyde concentration compensation value of the formaldehyde sensor 43 according to the received temperature information and/or humidity information, and to determine a target formaldehyde concentration value of the formaldehyde sensor 43 according to the formaldehyde concentration compensation value and the received initial formaldehyde concentration value.

In other embodiments of the present disclosure, the temperature information is a temperature value, the humidity information is a humidity value, and the processor 41 includes:
a difference calculation module configured to calculate a first difference between the temperature value and a set temperature threshold, and/or calculating a second difference between the humidity value and a set humidity threshold; and
a compensation value determination module configured to determine the formaldehyde concentration compensation value of the formaldehyde sensor 43 according to the first difference and/or the second difference calculated by the difference calculation module.

In other embodiments of the present disclosure, the compensation value determination module includes:
a first compensation value determination unit configured to determine the first formaldehyde concentration compensation value of the formaldehyde sensor 43 corresponding to the first difference between the temperature value and the set temperature threshold, according to a preset corresponding relationship between the difference, between the temperature value and the set temperature threshold, and the formaldehyde concentration compensation value of the formaldehyde sensor 43; and/or
a second compensation value determination unit configured to determine the second formaldehyde concentration compensation value of the formaldehyde sensor 43 corresponding to the second difference between the humidity value and the set humidity threshold, according to a preset corresponding relationship between the difference, between the humidity value and the set humidity threshold, and the formaldehyde concentration compensation value of the formaldehyde sensor 43; and
a formaldehyde concentration compensation value determination unit configured to set the first formaldehyde concentration compensation value determined by the first compensation value determination unit, or the second formaldehyde concentration compensation value determined by the second compensation value determination unit, or a sum of the first formaldehyde concentration compensation value determined by the first compensation value determination unit and the second formaldehyde concentration compensation value determined by the second compensation value determination unit, as the formaldehyde concentration compensation value of the formaldehyde sensor 43.

In other embodiments of the present disclosure, the processor 41 further includes
a parameter information acquisition module configured to acquire operating parameter information of the formaldehyde sensor 43 in a preset time period. The operating parameter information includes: total operating time period of the formaldehyde sensor 43 in the preset time period, and/or total number of operations of the formaldehyde sensor 43 in the preset time period.

The processor 41 sends a corresponding command signal to the formaldehyde sensor 43 in order to instruct the formaldehyde sensor 43 to perform zero point calibration, on a condition that the operating parameter information acquired by the parameter information acquisition module satisfies a preset condition.

In other embodiments of the present disclosure, the processor 41 further includes:
a first comparison module configured to compare the total operating time period of the formaldehyde sensor 43 in the preset time period with a set total operating time period threshold to obtain a first comparison result; and/or,
a second comparison module configured to compare the total number of operations of the formaldehyde sensor 43 in the preset time period with a set total number threshold of operations to obtain a second comparison result.

The processor 41 determines that the operating parameter information acquired by the parameter information acquisition module satisfies the preset condition, on a condition that the first comparison result obtained by the first comparison module is: the total operating time period of the formaldehyde sensor 43 in the preset time period is greater than the set total operating time period threshold, and/or on a condition that the second comparison result obtained by the second comparison module is: the total number of operations of the formaldehyde sensor 43 in the preset time period is greater than the set total number threshold of operations.

Some embodiments of the present disclosure also provide an air purifier, including the formaldehyde concentration measurement apparatus as shown in FIG. 4.

For the convenience of description, the above apparatus is described by illustrating various units that are divided according to their functions. Certainly, when implementing the present disclosure, the functions of the units can be implemented in the same one or more software and/or hardware components.

The various embodiments in this specification are described in a progressive manner, and the same or similar parts between the various embodiments can be referred to each other. Each embodiment focuses on the differences from other embodiments. In particular, for the apparatus or system embodiment, since it is basically similar to the method embodiment, the description is relatively simple, and the relevant part can refer to the part of the description of the method embodiment. The above-described apparatus and system embodiments are merely illustrative, wherein the units described as separate components may or may not be physically separated, and the components shown as units may or may not be physical units, i.e., they may be located in one place, or may be distributed to multiple network units. Some or all of the modules can be selected according to actual needs to achieve the objectives of the solutions of the embodiments. The present disclosure can be understood and implemented without creative work by those of ordinary skill in the art.

It should be noted that, relational terms such as “first” and “second” herein are only used to distinguish one entity or operation from another entity or operation, and do not necessarily require or imply that there is any such actual relationship or sequence between entities or operations. Moreover, the terms “include”, “comprise” or any other variants thereof are intended to cover non-exclusive inclusion, so that a process, method, product or device including a series of elements not only includes those elements, but also includes those that are not explicitly listed, or the inherent elements of this process, method, product or device. Without further limitations, the element defined by the expression “including a . . . ” does not exclude the existence of other identical elements in the process, method, product or device that includes the element.

The above are only specific embodiments of the present disclosure, which enables those skilled in the art to understand or implement the present disclosure. Various modifications to these embodiments will be apparent to those skilled in the art, and the general principles defined herein can be implemented in other embodiments without departing from the spirit or scope of the present disclosure. Therefore, the present disclosure will not be limited to the embodiments shown herein, but should conform to the widest scope consistent with the principles and novel features disclosed herein.

What is claimed is:

1. A formaldehyde concentration measurement method, comprising:
determining an initial formaldehyde concentration value measured by a formaldehyde sensor;
acquiring temperature information of an environment surrounding the formaldehyde sensor, and determining a formaldehyde concentration compensation value of the formaldehyde sensor according to the temperature information; or acquiring humidity information of the environment surrounding the formaldehyde sensor, and determining the formaldehyde concentration compensation value of the formaldehyde sensor according to the humidity information; or acquiring the temperature information and the humidity information of the environment surrounding the formaldehyde sensor, and determining the formaldehyde concentration compensation value of the formaldehyde sensor according to the temperature information and the humidity information; and
determining a target formaldehyde concentration value of the formaldehyde sensor according to the formaldehyde concentration compensation value and the initial formaldehyde concentration value.

2. The method according to claim 1, wherein the temperature information is a temperature value, and the humidity information is a humidity value;
the determining the formaldehyde concentration compensation value of the formaldehyde sensor according to the temperature information comprises: calculating a first difference between the temperature value and a set temperature threshold, and determining the formaldehyde concentration compensation value of the formaldehyde sensor according to the first difference;
the determining the formaldehyde concentration compensation value of the formaldehyde sensor according to the humidity information comprises: calculating a second difference between the humidity value and a set humidity threshold, and determining the formaldehyde concentration compensation value of the formaldehyde sensor according to the second difference; and
the determining the formaldehyde concentration compensation value of the formaldehyde sensor according to the temperature information and the humidity information comprises: calculating the first difference between the temperature value and the set temperature threshold and the second difference between the humidity value and the set humidity threshold; and determining the formaldehyde concentration compensation value of the formaldehyde sensor according to the first difference and the second difference.

3. The method according to claim 2, wherein
the determining the formaldehyde concentration compensation value of the formaldehyde sensor according to the first difference comprises: determining a first formaldehyde concentration compensation value of the formaldehyde sensor corresponding to the first difference between the temperature value and the set temperature threshold, according to a preset corresponding relationship between the first difference, between the temperature value and the set temperature threshold, and the formaldehyde concentration compensation value of the formaldehyde sensor; and setting the first formaldehyde concentration compensation value as the formaldehyde concentration compensation value of the formaldehyde sensor;
the determining the formaldehyde concentration compensation value of the formaldehyde sensor according to the second difference comprises: determining a second formaldehyde concentration compensation value of the formaldehyde sensor corresponding to the second difference between the humidity value and the set humidity threshold, according to a preset corresponding relationship between the second difference, between the humidity value and the set humidity threshold, and the formaldehyde concentration compensation value of the formaldehyde sensor; and setting the second formaldehyde concentration compensation value as the formaldehyde concentration compensation value of the formaldehyde sensor;
the determining the formaldehyde concentration compensation value of the formaldehyde sensor according to the first difference and the second difference comprises: determining the first formaldehyde concentration compensation value of the formaldehyde sensor corresponding to the first difference between the temperature value and the set temperature threshold, according to the preset corresponding relationship between the first difference, between the temperature value and the set temperature threshold, and the formaldehyde concentration compensation value of the formaldehyde sensor; determining the second formaldehyde concentration compensation value of the formaldehyde sensor corresponding to the second difference between the humidity value and the set humidity threshold, according to the preset corresponding relationship between the second difference, between the humidity value and the set humidity threshold, and the formaldehyde concentration compensation value of the formaldehyde sensor; and setting a sum of the first formaldehyde concentration compensation value and the second formaldehyde concentration compensation value as the formaldehyde concentration compensation value of the formaldehyde sensor.

4. The method according to claim 1, further comprising:
acquiring operating parameter information of the formaldehyde sensor in a preset time period, wherein the operating parameter information comprises: total operating time period of the formaldehyde sensor in the preset time period, or total number of operations of the formaldehyde sensor in the preset time period, or the total operating time period of the formaldehyde sensor in the preset time period and the total number of operations of the formaldehyde sensor in the preset time period; and
sending a corresponding command signal to the formaldehyde sensor in order to instruct the formaldehyde sensor to perform zero point calibration when the operating parameter information satisfies a preset condition.

5. The method according to claim 4, wherein the sending the corresponding command signal to the formaldehyde sensor in order to instruct the formaldehyde sensor to perform the zero point calibration, when the operating parameter information satisfies the preset condition, comprises:
when the operating parameter information of the formaldehyde sensor in the preset time period is the total operating time period of the formaldehyde sensor in the preset time period, comparing the total operating time period of the formaldehyde sensor in the preset time period with a set total operating time period threshold to obtain a first comparison result; determining that the operating parameter information satisfies the preset condition when the first comparison result is that the total operating time period of the formaldehyde sensor in the preset time period is greater than the set total operating time period threshold;
when the operating parameter information of the formaldehyde sensor in the preset time period is the total number of operations of the formaldehyde sensor in the preset time period, comparing the total number of operations of the formaldehyde sensor in the preset time period with a set total number threshold of operations to obtain a second comparison result; determining that the operating parameter information satisfies the preset condition when the second comparison result is that the total number of operations of the formaldehyde sensor in the preset time period is greater than the set total number threshold of operations;
when the operating parameter information of the formaldehyde sensor in the preset time period comprises the total operating time period of the formaldehyde sensor in the preset time period and the total number of operations of the formaldehyde sensor in the preset time period, comparing the total operating time period of the formaldehyde sensor in the preset time period with the set total operating time threshold to obtain the first comparison result, and comparing the total number of operations of the formaldehyde sensor in the preset time period with the set total number threshold of operations to obtain the second comparison result; and determining that the operating parameter information satisfies the preset condition when the first comparison result is that the total operating time period of the formaldehyde sensor in the preset time period is greater than the set total operating time period threshold, and the second comparison result is that the total number of operations of the formaldehyde sensor in the preset time period is greater than the set total number threshold of operations.

6. A formaldehyde concentration measurement apparatus, comprising:
a processor;
a communication interface;
a formaldehyde sensor electrically connected to the processor via the communication interface;
a temperature sensor, or a humidity sensor, or the temperature sensor and the humidity sensor, electrically connected to the processor via the communication interface,
wherein the formaldehyde sensor is configured to measure a concentration of formaldehyde to obtain an initial formaldehyde concentration value, and to send the initial formaldehyde concentration value to the processor;
the temperature sensor is configured to collect temperature information of an environment surrounding the formaldehyde sensor, and to send the temperature information to the processor;
the temperature sensor is configured to collect humidity information of the environment surrounding the formaldehyde sensor, and to send the humidity information to the processor;
when the apparatus comprises the temperature sensor, the processor is configured to determine a formaldehyde concentration compensation value of the formaldehyde sensor according to the temperature information that is received;
when the apparatus comprises the humidity sensor, the processor is configured to determine the formaldehyde concentration compensation value of the formaldehyde sensor according to the humidity information that is received;
when the apparatus comprises the temperature sensor and the humidity sensor, the processor is configured to determine the formaldehyde concentration compensation value of the formaldehyde sensor according to the temperature information and humidity information that are received; and
the processor is configured to determine a target formaldehyde concentration value of the formaldehyde sensor according to the formaldehyde concentration compensation value and the initial formaldehyde concentration value that is received.

7. The apparatus according to claim 6, wherein the temperature information is a temperature value, the humidity information is a humidity value, and the processor comprises a difference calculation module and a compensation value determination module;
when the apparatus comprises the temperature sensor, the difference calculation module is configured to calculate a first difference between the temperature value and a set temperature threshold, the compensation value determination module is configured to determine the formaldehyde concentration compensation value of the formaldehyde sensor according to the first difference calculated by the difference calculation module;
when the apparatus comprises the humidity sensor, the difference calculation module is configured to calculate a second difference between the humidity value and a set humidity threshold, the compensation value determination module is configured to determine the formaldehyde concentration compensation value of the formaldehyde sensor according to the second difference calculated by the difference calculation module;
when the apparatus comprises the temperature sensor and the humidity sensor, the difference calculation module is configured to calculate the first difference between the temperature value and the set temperature threshold and the second difference between the humidity value and the set humidity threshold, the compensation value determination module is configured to determine the formaldehyde concentration compensation value of the formaldehyde sensor according to the first difference and the second difference calculated by the difference calculation module.

8. The apparatus according to claim 7, wherein the compensation value determination module comprises:
a first compensation determination unit, or a second compensation determination unit, or the first compensation determination unit and the second compensation determination unit; and
a formaldehyde concentration compensation value determination unit;
when the difference calculation module is configured to calculate the first difference between the temperature value and the set temperature threshold, the compensation value determination module comprises the first compensation determination unit;
when the difference calculation module is configured to calculate the second difference between the humidity value and the set humidity threshold, the compensation value determination module comprises the second compensation determination unit;
when the difference calculation module is configured to calculate the first difference between the temperature value and the set temperature threshold and the second difference between the humidity value and the set humidity threshold, the compensation value determination module comprises the first compensation determination unit and the second compensation determination unit;
the first compensation value determination unit is configured to determine a first formaldehyde concentration compensation value of the formaldehyde sensor corresponding to the first difference between the temperature value and the set temperature threshold, according to a preset corresponding relationship between the first difference, between the temperature value and the set temperature threshold, and the formaldehyde concentration compensation value of the formaldehyde sensor;
the second compensation value determination unit is configured to determine a second formaldehyde concentration compensation value of the formaldehyde sensor corresponding to the second difference between the humidity value and the set humidity threshold, according to a preset corresponding relationship between the second difference, between the humidity value and the set humidity threshold, and the formaldehyde concentration compensation value of the formaldehyde sensor;
when the difference calculation module is configured to calculate the first difference between the temperature value and the set temperature threshold, the formaldehyde concentration compensation value determination unit is configured to set the first formaldehyde concentration compensation value determined by the first compensation value determination unit as the formaldehyde concentration compensation value of the formaldehyde sensor;
when the difference calculation module is configured to calculate the second difference between the humidity value and the set humidity threshold, the formaldehyde concentration compensation value determination unit is configured to set the second formaldehyde concentration compensation value determined by the second compensation value determination unit as the formaldehyde concentration compensation value of the formaldehyde sensor; and
when the difference calculation module is configured to calculate the first difference between the temperature value and the set temperature threshold and the second difference between the humidity value and the set humidity threshold, the formaldehyde concentration compensation value determination unit is configured to set a sum of the first formaldehyde concentration compensation value determined by the first compensation value determination unit and the second formaldehyde concentration compensation value determined by the second compensation value determination unit, as the formaldehyde concentration compensation value of the formaldehyde sensor.

9. The apparatus according to claim 6, wherein the processor further comprises:
a parameter information acquisition module configured to acquire operating parameter information of the formaldehyde sensor in a preset time period, wherein the operating parameter information comprises: total operating time period of the formaldehyde sensor in the preset time period, or total number of operations of the formaldehyde sensor in the preset time period, or the total operating time period of the formaldehyde sensor in the preset time period and the total number of operations of the formaldehyde sensor in the preset time period;
the processor is configured to send a corresponding command signal to the formaldehyde sensor in order to instruct the formaldehyde sensor to perform zero point calibration when the operating parameter information acquired by the parameter information acquisition module satisfies a preset condition.

10. The apparatus according to claim 9, wherein the processor further comprises a first comparison module, or a second comparison module, or the first comparison module and the second comparison module;
when the operating parameter information comprises the total operating time period of the formaldehyde sensor in the preset time period, the processor further comprises the first comparison module; when the operating parameter information comprises the total number of operations of the formaldehyde sensor in the preset time period, the processor further comprises the second comparison module; when the operating parameter information comprises the total operating time period of the formaldehyde sensor in the preset time period and the total number of operations of the formaldehyde sensor in the preset time period, the processor further comprises the first comparison module and the second comparison module;
the first comparison module is configured to compare the total operating time period of the formaldehyde sensor in the preset time period with a set total operating time period threshold to obtain a first comparison result;
the second comparison module is configured to compare the total number of operations of the formaldehyde sensor in the preset time period with a set total number threshold of operations to obtain a second comparison result;
when the processor further comprises the first comparison module, and when the first comparison result obtained by the first comparison module is that the total operating time period of the formaldehyde sensor in the preset time period is greater than the set total operating time period threshold, the processor determines that the operating parameter information acquired by the parameter information acquisition module satisfies the preset condition;
when the processor further comprises the second comparison module, and when the second comparison result obtained by the second comparison module is that the total number of operations of the formaldehyde sensor in the preset time period is greater than the set total number threshold of operations, the processor determines that the operating parameter information acquired by the parameter information acquisition module satisfies the preset condition;
when the processor further comprises the first comparison module and the second comparison module, and when the first comparison result obtained by the first comparison module is that the total operating time period of the formaldehyde sensor in the preset time period is greater than the set total operating time period threshold, and the second comparison result obtained by the second comparison module is that the total number of operations of the formaldehyde sensor in the preset time period is greater than the set total number threshold of operations, the processor determines that the operating parameter information acquired by the parameter information acquisition module satisfies the preset condition.

11. An air purifier, comprising the formaldehyde concentration measurement apparatus according to claim 6.
